(19) United States
(12) Patent Application Publication
(10) Pub. No.: US 2025/0025355 A1
(43) Pub. Date: Jan. 23, 2025

(54) ABSORBENT CORE AND ABSORBENT ARTICLE COMPRISING THE SAME

(71) Applicant: The Procter & Gamble Company, Cincinnati, OH (US)

(72) Inventors: Fengchun YE, BEIJING (CN); Yimeng WANG, BEIJING (CN); Zheng BAO, BEIJING (CN)

(21) Appl. No.: 18/777,663

(22) Filed: Jul. 19, 2024

(30) Foreign Application Priority Data
Jul. 20, 2023 (WO) PCT/CN2023/108270

(51) Int. Cl. A61F 13/537; A61F 13/53; A61F 13/539

(52) U.S. Cl. CPC A61F 13/53752; A61F 13/539; A61F 2013/53051

(57) ABSTRACT

The present invention is directed to an absorbent core for use in an absorbent article and an absorbent article comprising the same, the absorbent core comprising a liquid-permeable top cover layer; a bottom cover layer; and an absorbent layer disposed between the top cover layer and the bottom cover layer, the absorbent layer comprising superabsorbent polymer particles; and wherein the top cover layer comprises pulp, and has an MD tensile strength at least about 12N, wherein the top cover layer has a first MD wicking length at 3 min, and the bottom cover layer has a second MD wicking length at 3 min as measured according to Wicking Length Test, wherein the first MD wicking length is at least about 70 mm, and at least about 3 times the second MD wicking length.

### CROSS REFERENCE TO RELATED APPLICATION

This application is a continuation, and claims priority under 35 U.S.C. § 119 (b), of Patent Application No. PCT/CN2023/108270, filed on Jul. 20, 2023, which is hereby incorporated by reference herein in its entirety.

### FIELD OF THE INVENTION

The invention relates to absorbent cores and their use in personal hygiene absorbent articles.

### BACKGROUND OF THE INVENTION

Absorbent articles for personal hygiene such as disposable baby diapers, training pants for toddlers or adult incontinence undergarments, are designed to absorb and contain body exudates, in particular urine. These absorbent articles comprise several layers providing different functions, typically including a topsheet, a backsheet and an absorbent core in-between, among other layers.

The absorbent core should be able to absorb and retain the exudates for a prolonged amount of time, for example overnight for a diaper, minimize re-wet to keep the wearer dry, and avoid soiling of clothes or bed sheets. Absorbent cores have typically comprised a blend of comminuted wood pulp cellulose fibers with superabsorbent polymers (SAP) particles, also called absorbent gelling materials (AGM), as absorbent material.

Absorbent cores without fluff cellulose fibers (also called “airfelt-free” cores) have been more recently proposed. The SAP particles may be for example enclosed within discrete pockets. It has been also proposed to immobilize SAP particles with a microfibrous adhesive network to a nonwoven substrate by an adhesive (see e.g. WO2008/155699A1, Hundorf et al.). WO 2021/132295A discloses an absorbent sheet comprising a first fiber sheet, a second fiber sheet, and absorbent polymers disposed between these fiber sheets where Klemm water absorption heights of the first fiber sheet is higher than the Klemm water absorption height of the second fiber sheet.

More recently, airfelt-free cores comprising a high loft, fibrous, central nonwoven sheet with SAP at least partially distributed within this central high loft nonwoven have been disclosed (see e.g. WO2016/106,021A1, Bianchi et al.). SAP particles are applied on one or both sides of the high loft nonwoven, with the SAP particles deposited on the surface of the central nonwoven sheet being at least partially distributed and immobilized within the pores of the central nonwoven sheet. A nonwoven is further adhesively attached on each of the high loft central nonwoven sheet to further immobilize the particles within the high loft central nonwoven sheet. At least the top cover layer which is planned to be oriented towards the topsheet on the absorbent article should be fluid-pervious. A further wrap layer may be typically used to further stabilize these layers and form the absorbent core. Disclosures of this type of absorbent cores include WO2020/025401 (BASF, Ge et al.), WO2020/032280, WO2020/032281, WO2020/032282, WO2020/032283 and WO2020/032284 (Nippon SHOKUBAI).

There is a continuous need to improve the performances of absorbent cores, in particular in terms of absorption speed, absorption capacity, low rewet and/or wearer comfort, while keeping the overall costs of manufacture as low as possible.

### SUMMARY OF THE INVENTION

The present invention is directed to an absorbent core for use in an absorbent article, the absorbent core extending in a transversal direction and a longitudinal direction, the absorbent core comprising a liquid-permeable top cover layer; a bottom cover layer; and an absorbent layer disposed between the top cover layer and the bottom cover layer, the absorbent layer comprising superabsorbent polymer particles, wherein the top cover layer comprises pulp, and has an MD tensile strength at least about 12N, wherein the top cover layer has a first MD wicking length at 3 min, and the bottom cover layer has a second MD wicking length at 3 min as measured according to Wicking Length Test, wherein the first MD wicking length is at least about 70 mm, and at least about 3 times the second MD wicking length.

The present invention is also directed to an absorbent core for use in an absorbent article, the absorbent core extending in a transversal direction and a longitudinal direction, the absorbent core comprising a liquid-permeable top cover layer; a bottom cover layer; and an absorbent layer comprising superabsorbent polymer particles, wherein the top cover layer comprises first absorbent fibers and second absorbent fibers, the second absorbent fibers being different from the first absorbent fibers, and wherein the total amount of the first absorbent fibers and the second absorbent fibers is at least about 70% by weight of the top cover layer.

The present invention is directed to an absorbent article comprising a topsheet, a backsheet, and an absorbent core disclosed herein and disposed between the topsheet and the backsheet.

The article is illustrated in the Figures as a taped diaper. For ease of discussion, the absorbent article and the acquisition-distribution system will be discussed with reference to the numerals referred to in these Figures. The Figures and detailed description should however not be considered limiting the scope of the claims, unless explicitly indicated otherwise. In particular, the invention may also be used in a wide variety of absorbent article forms, such as pant type diapers, which are pre-formed and are worn like an underwear garment, or female protection sanitary pads.

### BRIEF DESCRIPTION OF THE DRAWINGS

FIG. 1 shows a top view of an exemplary absorbent core with the top and central nonwoven sheets partially removed.

FIG. 2 shows a schematic cross-sectional view of an absorbent core.

FIG. 3 shows a schematic cross-sectional view of an alternative absorbent core.

FIG. 4 shows a schematic cross-sectional view of an alternative absorbent core.

FIG. 5 shows a schematic cross-sectional view of an alternative absorbent core.

FIG. 6 shows a schematic cross-sectional view of an absorbent core as in FIG. 3 with a core wrapping layer.

FIG. 7 shows a schematic cross-sectional of an alternative absorbent core comprising two central nonwoven sheets.

FIG. 8 is a schematic sketch of a process for making a wetlaid nonwoven.

FIG. 9 shows a schematic cross-sectional of an absorbent article comprising the absorbent core of FIG. 6.

FIG. 10 is a schematic sketch of a process for making an absorbent core of the invention.

FIG. 11A and FIG. 11B is to illustrate the Wicking Area Test.

FIG. 12 illustrates an apparatus used in the Modified Fluid Acquisition Test.

FIG. 13A is a side view of the curved component used in the Modified Fluid Acquisition Test.

FIG. 13B is an end view of the curved component of FIG. 13A.

FIG. 13C is a bottom view of the curved component of FIG. 13A.

FIG. 13D is a bottom perspective view of the curved component of FIG. 13A.

FIG. 13E is a top perspective view of the curved component of FIG. 13A.

FIG. 14A illustrates a top plate assembly used in the Modified Fluid Acquisition Test.

FIG. 14B illustrates equipment used in the Modified Fluid Acquisition Test.

### DETAILED DESCRIPTION OF THE INVENTION

### Definitions of Terms

The term “absorbent article” as used herein refers to disposable products such as taped diapers, diapers having a closed waist opening (pants), feminine hygiene sanitary napkins and the like, which are placed against or in proximity to the body of the wearer to absorb and contain bodily exudates such as urine, feces and menses discharged from the body. Typical absorbent articles comprise a topsheet, a backsheet, an absorbent core, an acquisition layer and other components. A liquid permeable topsheet forms at least a portion of the wearer-facing side of the article, and a backsheet forms at least a portion, and typically the whole, of the garment-side of the article. The articles may be provided with fastening elements, such as tapes (taped diapers) or may be provided already pre-formed with a waist opening and a pair of leg openings as in an underwear (pant diapers). The absorbent articles may be for use with babies, infants, women or incontinent adults. Typical features of absorbent articles are further discussed further below.

As used herein, the term “cellulose fibers” intends to include both natural cellulose fibers such as pulp and cotton, and regenerated cellulose fibers such as rayon (including viscose, lyocell, MODAL (a product of Lenzing AG, Lenzing, Austria) and cuprammonium rayon) unless specified differently.

The term “joined” or “bonded” or “attached”, as used herein, encompasses configurations whereby an element is directly secured to another element by affixing the element directly to the other element e.g. by gluing, and configurations whereby an element is indirectly secured to another element by affixing the element to intermediate member(s) which in turn are affixed to the other element.

The terms “comprise,” “comprising,” and “comprises” are open ended terms, each specifies the presence of the feature that follows, e.g. a component, but does not preclude the presence of other features, e.g. elements, steps, components known in the art or disclosed herein. These terms based on the verb “comprise” should be read as encompassing the narrower terms “consisting essential of” which excludes any element, step or ingredient not mentioned which materially affect the way the feature performs its function, and the term “consisting of” which excludes any element, step, or ingredient not specified. Any preferred or exemplary examples described below are not limiting the scope of the claims, unless specifically indicated to do so. The words “typically”, “normally”, “advantageously” and the likes also qualify features which are not intended to limit the scope of the claims unless specifically indicated to do so.

The terms “machine direction” or “MD” is the direction parallel to the direction of travel of the web in a manufacturing process. The machine direction is typically the longitudinal direction of a component of an absorbent article. The “cross machine direction” or “CD” is the direction substantially perpendicular to the MD and in the plane generally defined by the web.

The terms “nonwoven”, nonwoven layer” or “nonwoven web” are used interchangeably to mean an engineered fibrous assembly, primarily planar, which has been given a designed level of structural integrity by physical and/or chemical means, excluding weaving, knitting or papermaking (ISO 9092:2019 definition). The directionally or randomly orientated fibers, are bonded by friction, and/or cohesion and/or adhesion. The fibers may be of natural or synthetic origin and may be staple or continuous filaments or be formed in situ. Commercially available fibers have diameters ranging from less than about 0.001 mm to more than about 0.2 mm and they come in several different forms such as short fibers (known as staple, or chopped), continuous single fibers (filaments or monofilaments), untwisted bundles of continuous filaments (tow), and twisted bundles of continuous filaments (yam). Nonwoven webs can be formed by many processes such as meltblowing, spunbonding, solvent spinning, electrospinning, carding and airlaying. The basis weight of nonwoven webs is usually expressed in grams per square meter (g/m2 or gsm).

### Absorbent Core

As used herein, the term “absorbent core” refers to a component for an absorbent article comprising an absorbent material that can absorb and retain body fluid such as urine and menstrual blood.

By “absorbent material” it is meant a material which has some absorbency property or liquid retaining properties, such as SAP, cellulosic fibers as well as synthetic fibers. Typically, glues used in making absorbent cores have no absorbency properties and are not considered as absorbent material. The core wrap is not considered as absorbent material for the purpose of assessing the percentage of SAP in the absorbent core.

The absorbent cores according to this invention may be manufactured in a continuous stream that can be stored and transported for example as a roll of absorbent core material, and are then individualized when integrated in an absorbent article, such as a diaper and a sanitary napkin. Absorbent cores have the most absorbent capacity of the components of the absorbent article and comprises all, or at least the majority of, SAP particles. The terms “absorbent core” and “core” are herein used interchangeably.

The absorbent cores of the invention may be substantially planar. By substantially planar, it is meant that the absorbent core can be laid flat on a planar surface and primarily extend in an x and an y direction. The absorbent cores may also be typically thin and conformable, so that they can also be laid on a curved surface for example a drum during the making process, or stored and handled as a continuous roll of stock material comprising a plurality of cores before being converted into an absorbent article.

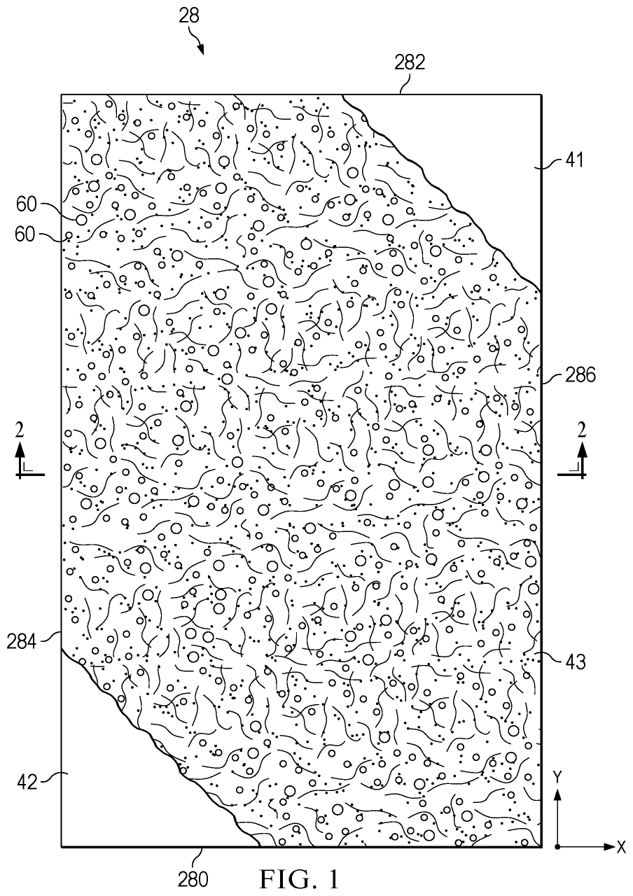

An exemplarily individualized absorbent core is represented in a flat state of FIG. 1. Though the absorbent core 28 in FIG. 1 is in a rectangle shape having a front edge 280, a back edge 282 and two longitudinally-extending side edges 284, 286, the absorbent core of the present invention can be in a variety of shapes meeting various needs.

The absorbent core's height in the z direction is small relative to its other dimensions in the transversal direction x and the longitudinal direction y. Unless otherwise indicated, dimensions and areas disclosed herein apply to the core in this flat-out configuration.

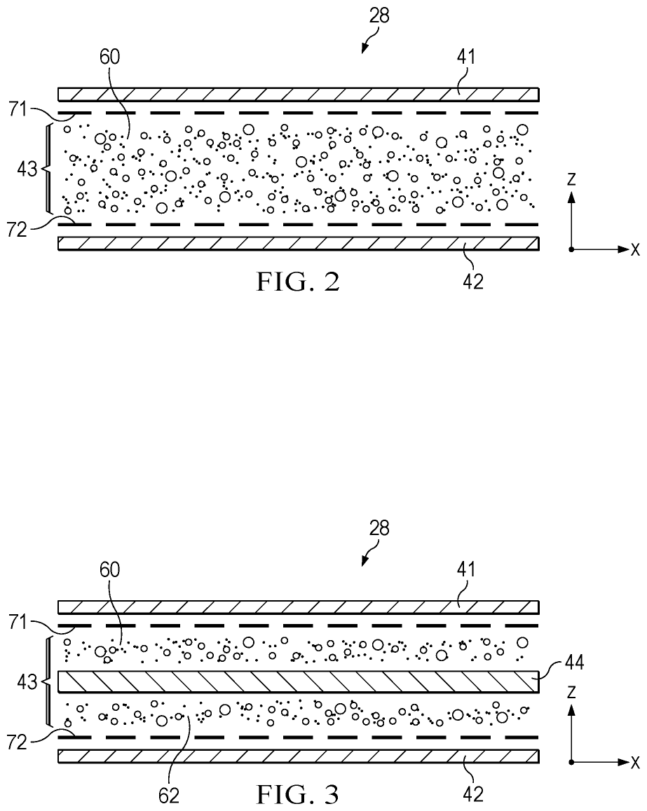

Referring to FIGS. 2 and 3, the absorbent core of the invention comprises a liquid-permeable top cover layer 41, a bottom cover layer 42, and an absorbent layer 43 disposed between the top cover layer 41 and the bottom cover layer 42, the absorbent layer comprising an SAP 60. Referring to FIG. 2, the SAP is deposited at least one of an inner surface of the top cover layer 41 and an inner surface of the bottom cover layer 42.

Referring to FIG. 3, the absorbent layer may further comprise a central nonwoven sheet 44 having a top surface oriented towards the top cover layer 41 and a bottom surface oriented towards the bottom cover layer 42, wherein the SAP is deposited on at least one of the top surface and the bottom surface of the central nonwoven sheet 44.

In the first embodiment of the present invention, the absorbent core of the invention comprises a top cover layer a first MD wicking length at 3 min as measured according to Wicking Length Test, and a bottom cover layer has a second MD wicking length at 3 min as measured according to Wicking Length Test, the first MD wicking length being at least 3 times, or at least 4 times, or at least 5 times greater than the second MD wicking length. With the significantly higher MD wicking length of the top cover layer than the bottom cover, the absorbent core of the present invention can draw down the fluid from a topsheet quickly and effectively and drives the topsheet dryness, and utilize SAP in the absorbent core better.

In the second embodiment of the present invention, the top cover layer comprises first absorbent fibers and second absorbent fibers which differ from the first absorbent fibers, and the total amount of the first absorbent fibers and the second absorbent fibers is at least about 70% by weight, or at least about 80% by weight, or at least about 90% by weight of the top cover layer.

For case of discussion, the absorbent cores, articles and processes of the invention will be discussed with reference to the Figures and the numerals referred to in these Figures; however these are not intended to limit the scope of the claims unless specifically indicated.

### Absorbent Layer

Referring to FIGS. 2 and 3, the absorbent core of the present invention comprises an absorbent layer 43 comprising superabsorbent polymer particles 60, 62, 64. The absorbent layer 43 is disposed between a top cover layer 41 and the bottom cover layer 42.

Referring to FIGS. 3-7, the absorbent layer may further comprise a central nonwoven sheet 44 having a top surface oriented towards the top cover layer 41 and a bottom surface oriented towards the bottom cover layer 42, wherein the superabsorbent polymer particles 60, 62, 64 is deposited on at least one of the top surface and the bottom surface of the central nonwoven sheet 44.

The superabsorbent polymer particle may be distributed homogeneously in the core.

Alternatively, it can be profiled, with higher amount of SAP towards the front half of the core relative to the back half of the core. This is because there is typically more fluid discharged towards the front of the article in which a core will be incorporated. In addition to the profiled SAP distribution in the longitudinal direction (y), the SAP may be also profiled in the transversal direction (x). Typically, the SAP is however homogenously distributed in the transversal (x) and the longitudinal direction (y), which simplifies production: in that case any of the two shorter sides may be considered as the front edge and the opposite side will be the back edge.

The absorbent layer may be substantially free of free cellulose fibers which are not integrated with the other fibers of nonwoven. By “substantially free of free cellulose fibers”, it is meant that the amount of the free cellulose fibers may be less than 10% by weight of the absorbent layer, or less than 5% by weight of the absorbent layer, or less than 1% by weight of the absorbent layer, or completely free of such free cellulose fibers.

### Top Cover Layer and Bottom Cover Layer

The top cover layer 41 is on the side of the absorbent core intended to be placed closest to the wearer-facing side of the absorbent article and may be liquid-permeable. The bottom cover layer is positioned on the other side of the central nonwoven sheet. It may be liquid-permeable or liquid impermeable. The top cover layer and the bottom cover layer provide a cover on both sides of the central nonwoven sheet for preventing the SAP particles from falling out of the central nonwoven sheet during the core and article making process and/or during use of the absorbent article.

In the first embodiment of the present invention, the first MD wicking length is at least about 70 mm, or at least about 80 mm, or at least about 90 mm, or at least about 100 mm.

The top cover layer has a first MD wicking length at 3 min as measured according to Wicking Length Test, and the bottom cover layer has a second MD wicking length at 3 min as measured according to Wicking Length Test, and the first MD wicking length may be at least about 3 times, or at least about 5 times, or at least about 7 times, or at least 10 times greater than the second MD wicking length.

With the high first MD wicking length and the significantly higher MD wicking length of the top cover layer than the bottom cover, the absorbent core of the present invention can draw down the fluid from a topsheet quickly and effectively and drives the topsheet dryness, and utilize SAP in the absorbent core better.

Nonwovens materials are typically inherently hydrophobic, and optionally, the top cover layer or bottom cover layer may be treated differently to render to the top cover layer more hydrophilic than the bottom cover layer.

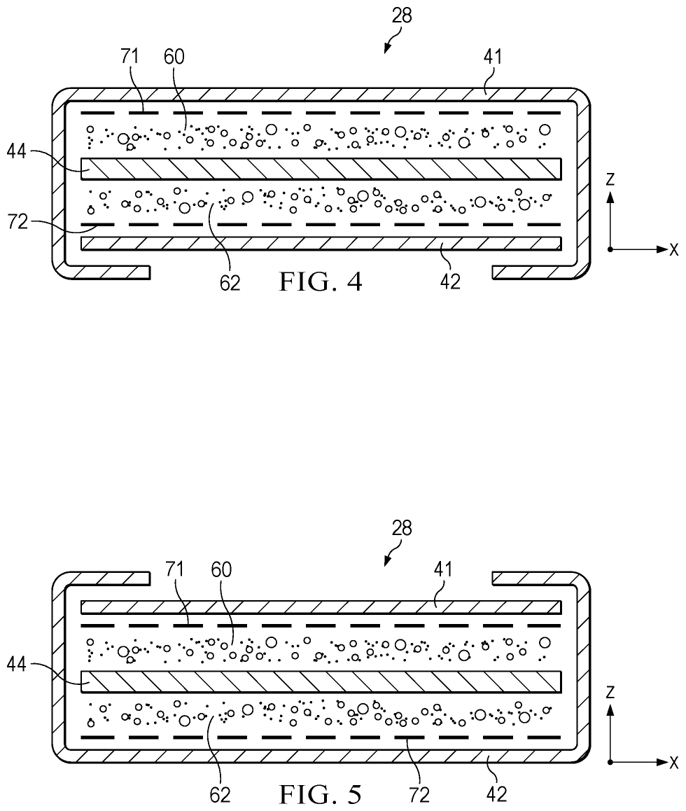

The top cover layer 41 can be wider than the bottom cover layer 42 so that this excess material can be folded around the longitudinal side edges 284, 286 of the core 28 to form a C-wrap seal over the bottom cover layer 42, as illustrated in FIG. 4. Alternatively, the bottom cover layer 42 can be wider than the top cover layer 41 so that this excess material can be folded around the longitudinal side edges 284, 286 of the core 28 to form a C-wrap seal over the top cover layer 41, as illustrated in FIG. 5.

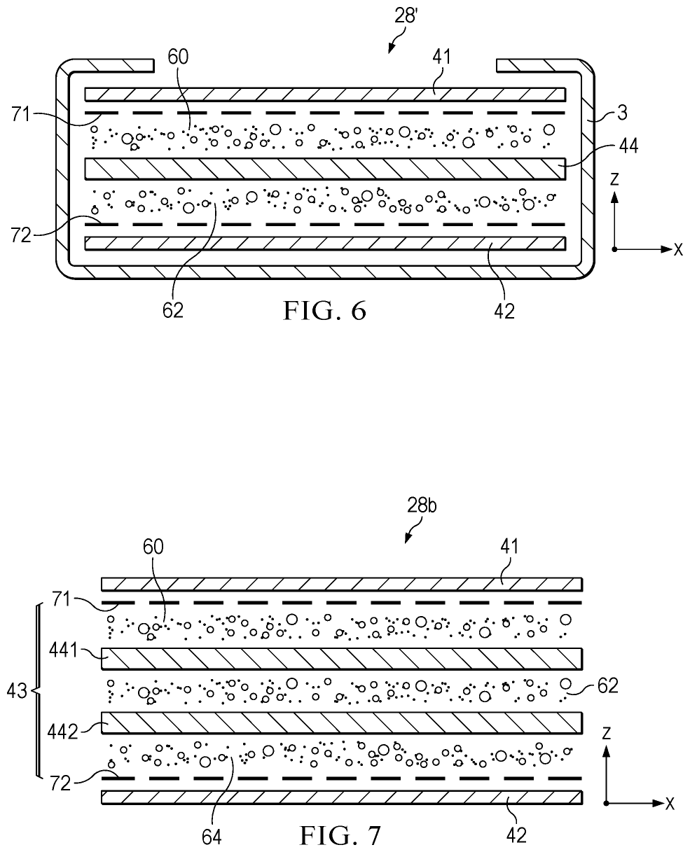

In addition to the top cover layer and the bottom cover layer, the absorbent core can further comprise a wrapping layer 3 that encompasses the central nonwoven sheet and the two cover layers, such by forming a C-wrap around the longitudinally extending side edges 284, 286 of the core, as shown in FIG. 6. By “C-wrap”, it is meant that the layer covers at least the top side or bottom side of the core, extends along its side edges to form flaps that are then folded and attached, typically by gluing, over the opposite side of the core. The wrapping layer 3 may thus have a cross-section similar to the letter C (when rotated) 90°. A C-wrap construction may further help containing the SAP particles during the making or wearing of the absorbent article. The wrapping layer may for example be made of a low basis weight nonwoven layer, for example having a basis weight of from 5 to 40 gsm, in particular from 8 to 25 gsm, in particular a SMS nonwoven, but other materials are of course possible. As one example, the wrapping layer 3 may extends from the bottom side of the core 28 and having flaps folded over the top side of the core as represented in FIG. 6. The inverted configuration is also possible, with the C-wrapped layer 3 extending from the top side and with the flaps folded over the bottom side. The folded flaps may end and be attached in the vicinity of the longitudinally extending side edges of the core or may be longer than represented to that they overlap and attached to another. The presence of a wrapping layer is optional though is preferred especially if the top cover layer and the bottom cover layer are not sealed along their longitudinal sides.

The top cover layer 41 and/or the bottom cover layer 42 are preferably attached at least partially to the absorbent layer 43 or the central nonwoven sheet 44.

A layer of glue 71 may be for example applied between the top cover layer 41 and the absorbent layer 43. Any type of conventional glue and glue application method may be used. Typically, a hot melt glue may be sprayed on substantially the whole of the surface of the layers before putting the two layers in close contact so that they become attached. The glue may also be applied by a contact method to one of the layers, in this case in particular the top or bottom cover layer, typically by slot-coating a series of parallel thin lines of glue in the machine direction (y direction). A layer of glue 72 may also be similarly applied between the bottom cover layer 42 and the absorbent layer 43. These layers of glue also have the advantages that they can immobilize the SAP particles in the dry state that have not penetrated within the central nonwoven sheet during the making of the core.

### Top Cover Layer

The top cover layer is thus liquid-permeable, so that a fluid can easily reach the absorbent layer through the top cover layer during use.

The top cover layer comprises absorbent fibers.

In the first embodiment of the present invention, the top cover layer comprises absorbent fibers comprising pulp. The top cover layer may comprise pulp in the range of about 30%-70%, or about 40%-60%, or about 30-60% by weight of the top cover layer.

When the amount of pulp is less than 30% by weight of the top cover layer, the top cover layer may not provide sufficient wicking power desired for the present invention, and it may also increase production cost given pulp needs to be more expensive absorbent fibers. When the amount of pulp is higher than 70% by weight of the top cover layer, the MD tensile strength of the top cover layer is low and does not have a sufficient mechanical strength.

The top cover layer may comprise absorbent fibers including pulp at least about 70%, at least about 80%, at least about 80% or at least about 90% by weight of the top cover layer. The top cover layer may comprise 100% absorbent fibers.

In the second embodiment of the present invention, the top cover layer comprises first absorbent fibers and second absorbent fibers which differ from the first absorbent fibers. The total amount of the first and second absorbent fibers is at least about 70%, at least about 80%, at least about 80% or at least about 90% by weight of the top cover layer. The top cover layer may comprise 100% absorbent fibers.

Absorbent fibers can provide absorption of liquid insults from the topsheet when the absorbent core of the present invention is a component in absorbent articles. Any suitable absorbent fibers may be utilized. Some examples of absorbent fibers are cellulose fibers including cotton, pulp, rayon or regenerated cellulose or combinations thereof.

The absorbent fibers suitable for the top cover layer may have any suitable shape. Some examples include trilobal, “H,” “Y,” “X,” “T,” round, or flat ribbon. Further, the absorbing fibers can be solid, hollow or multi-hollow.

When the top cover sheet comprising absorbent fibers especially pulp fibers absorbs liquid, it tends to lose a structural integrity to some degrees which results in weakening mechanical strength of the top cover sheet and eventually an absorbent core. It may reduce the resiliency of the absorbent article containing the absorbent core and lead to increased bunching and increased leakage risk in absorbent article during usage.

In order to address the potential problems associated with loss of structural integrity and mechanical strength illustrated above, the top cover layer disclosed herein may comprise first absorbent fibers and second absorbent fibers different from the first absorbent fibers. The first absorbent fibers may more contribute to high weaking power and the second absorbent fibers may more contribute to mechanical strength of the top cover layer. The first absorbent fibers may be pulp fibers. The second absorbent fibers may be viscose fibers. When the top cover layer comprises pulp, the amount of pulp may be in the range of about 30%-70%, or about 40%-60%, or about 30-60% by weight of the top cover layer.

In some examples, the top cover layer comprises pulp fibers at least about 30% and viscose fibers at least about 40% by weight for the tops cover layer.

The top cover layer may further comprise synthetic fibers. Optional synthetic fibers forming the top cover layer may be made partially or entirely of a relatively resilient synthetic fibers, in particular polypropylene (PP), polyamide (PA, such as nylons) or polyethylene terephthalate (PET) fibers. Synthetic fibers may impart the top cover layer a better MD tensile strength.

The top cover layer may have a first MD wicking length at least about 70 mm, at least about 80 mm, at least about 90 mm, or even at least 100 mm at 3 min as measured according to Wicking Length Test disclosed herein.

The top cover layer may have a first wicking area at least about 100 mm², at least about 150 mm², at least about 170 mm², or at least about 190 mm², as measured according to Wicking Area Test disclosed herein.

The top cover layer may be for example a nonwoven having a basis weight of between 5 gsm and 60 gsm, or between 10 gsm and 50 gsm, or between 20 gsm and 50 gsm.

The top cover layer may comprise wetlaid nonwoven. The top cover layer may consist of wetlaid nonwoven. The principle of wetlaying is similar to paper manufacturing. Referring to FIG. 8, a dilute slurry of water and fibers in a container 810 is deposited on a moving wire screen where the water is drained and the fibers form a web (820). The web can be further dewatered by pressing between rollers (830) and dried (850). Impregnation with binders (840) is often included in a later stage of the process.

The top cover layer may have a MD tensile strength of at least 12N, or at least about 14 N, or at least about 15N as measured according to Tensile Strength Test disclosed herein. Materials such as a tissue sheet having a MD tensile strength lower than 12N is not suitable for a top cover layer in the present invention as it does not have a sufficient mechanical strength to endure a core manufacturing process and an absorbent article manufacturing process. The top cover layer may have a MD tensile strength/basis weight at least about 1.5 N/gsm, or at least 1.8 N/gsm, or at least 2 N/gsm.

The top cover layer may be treated to render it hydrophilic, for example by treating it with a surfactant or other methods as is known in the art.

### Bottom Cover Layer

The bottom cover layers may be made of a relatively thin and cheap material, as are commonly used for the production of conventional cores. The bottom cover layer may be for example a nonwoven web having a basis weight of between 5 gsm and 50 gsm, such as a carded nonwoven, spunbond nonwoven (“S”) or meltblown nonwoven (“M”), and laminates of any of these. For example, spunmelt polypropylene nonwovens are suitable, in particular those having a laminate web SMS, or SMMS, or SSMMS, structure, and having a basis weight range of about 5 gsm to 20 gsm.

The bottom cover layer may have a second MD wicking length no greater than about 30 mm, or no greater than about 20 mm, or no greater than about 10 mm at 3 min as measured according to Wicking Length Test disclosed herein.

### Central Nonwoven Sheet

The absorbent cores of the invention may comprise a central nonwoven sheet 44, as illustrated in FIGS. 3-7. The central nonwoven sheet 44 is sandwiched between a top cover layer 41 and a bottom cover layer 42. The central nonwoven sheet 44 may comprise high loft nonwoven.

The term “high loft” refers to low density bulky fabrics, as compared to flat, paper-like fabrics. High loft webs are characterized by a relatively high porosity. This means that there is a relatively high amount of void space in which superabsorbent polymer particles can be distributed. The high loft nonwoven without SAP particles may have a density at a pressure of 4.14 kPa (0.6 psi) below 0.20 g/cm³, in particular ranging from 0.05 g/cm³ to 0.15 g/cm³, or a density at a pressure of 2.07 kPa (0.3 psi) below 0.20 g/cm³, in particular ranging from 0.02 g/cm³ to 0.15 g/cm³, or a density at a pressure of 0.83 kPa (0.12 psi) below 0.15 g/cm³, in particular ranging from 0.01 g/cm³ to 0.15 g/cm³.

The density can be calculated by dividing the basis weight of the high loft nonwoven by its thickness measured at the respective pressure according to Thickness and Density Test disclosed herein.

The central nonwoven sheet comprises preferably high loft nonwoven, but other types of high loft material are not excluded. The central nonwoven sheet may comprise or consist of synthetic fibers, optionally mixed with natural fibers such as cellulose or cotton fibers or viscose fibers for example. The central nonwoven sheet may be substantially free of free cellulose fibers which are not integrated with the other fibers of the nonwoven. The amount of such free cellulose fibers in the absorbent core may be less than 10% by weight of the total absorbent core, or less than 5% by weight of the total absorbent core, or less than 1% by weight of the total absorbent core, or completely free of such free cellulose fibers. The high loft material may comprise at least 10%, 30% 50%, 70%, 90% and up to 100% by weight of the central nonwoven sheet, of synthetic fibers.

The fibers forming the central nonwoven sheet may be made partially or entirely of a relatively resilient synthetic fibers, in particular polypropylene (PP), polyamide (PA, such as nylons) or polyethylene terephthalate (PET) fibers. The diameter of the fibers may for example range from 0.01 mm to 0.50 mm.

The thickness, basis weight and density of the central nonwoven sheet are typically homogenous in both transversal direction (x) and longitudinal direction (y). The orientation of fibers in the central nonwoven sheet may be in-homogenous such as predominant orientation of fibers into one direction x or y such as in carded nonwovens. Furthermore, the fiber orientation in the central nonwoven sheet in thickness direction z may be different versus the predominant orientation in one or both directions x and/or y.

The central nonwoven sheet may in particular have a thickness of at least 0.30 mm, in particular ranging from 0.30 mm to 2.00 mm, or from 0.50 mm to 1.5 mm, as measured at a pressure of 4.14 kPa (0.6 psi) according to the test method described further below. The central nonwoven sheet may in particular have a thickness ranging from 0.30 mm to 2.50 mm or from 0.5 to 2.0 mm or from 0.7 to 1.3 mm, as measured at a pressure of 0.83 kPa (0.12 psi) according to the test method described further below.

The basis weight of the central nonwoven sheet may for example range from 15 gsm to 500 gsm, in particular from 20 gsm to 200 gsm, more particularly of from 30 gsm to 100 gsm.

The values indicated herein for the central nonwoven sheet are considered for the high loft material taken in isolation, that is before the SAP particles have been deposited between the fibers or an adhesive applied to it, unless indicated otherwise. When the absorbent core comprises two or more central nonwoven sheets, these may be the same or different.

While the invention is not limited to a specific type of nonwoven or fibers, a particular example of suitable nonwoven as the central nonwoven sheet are bonded carded webs (“BCW”). “Bonded carded web” refers to nonwovens that are made from staple fibers that are sent through a combing or carding unit, which separates and generally aligns the staple fibers in the machine direction to form a generally machine direction-oriented fibrous nonwoven web. This web can then be drawn through a heated drum, creating bonds throughout the fabric without applying specific pressure (through air bonding process). Such through air bonded carded web (TABCW) material provides a low density, lofty through-air bonded carded web. Examples of suitable TABCW are for example disclosed in WO2000/71067 (KIM DOO-HONG et al.). The carded webs may also be bonded by other methods, such as mechanical entanglement of the fibers (needle punching for example).

In a carded nonwoven, the fibers in the web are aligned predominantly in the machine direction and have a more uniform fiber alignment than other nonwovens, which results in greater stability and internal bond strength especially in machine direction. The bonding technique chosen influences the integrity of the fabric. Through-air bonded carded web have excellent softness, bulk and compressibility, and rapid strike through and good rewet. Synthetic, natural and recycled fibers in a wide range of deniers can be used. Soft PE/PP bicomponent staple fibers may in particular be used. The carded nonwoven material can for example comprise about 3 to about 10 denier staple fibers.

The central nonwoven sheet may also comprise a spunmelt nonwoven. Spunmelt is a generic term describing the manufacturing of nonwoven webs directly from thermoplastic polymers. It encompasses two processes and the combination of both: spunlaid (also known as spunbond) nonwoven and meltblown nonwoven. In a spunlaid process, polymer granules are melted and molten polymer is extruded through spinnerets. The continuous filaments are cooled and deposited onto a conveyor to form a uniform web. Some remaining temperature can cause filaments to adhere to one another, but this cannot be regarded as the principal method of bonding. The spunlaid process has the advantage of giving nonwovens greater strength, but raw material flexibility is more restricted. Co-extrusion of second components is used in several spunlaid processes, usually to provide extra properties or bonding capabilities. In meltblown web formation, low viscosity polymers are extruded into a high velocity airstream on leaving the spinneret. This scatters the melt, solidifies it and breaks it up into a fibrous web.

When the central nonwoven sheet 44 is in a rectangle shape, a front and a back edges are typically shorter than side edges. The front edge of the central nonwoven sheet corresponds to the edge intended to be placed towards the front edge of the absorbent article in which the core is or will be integrated.

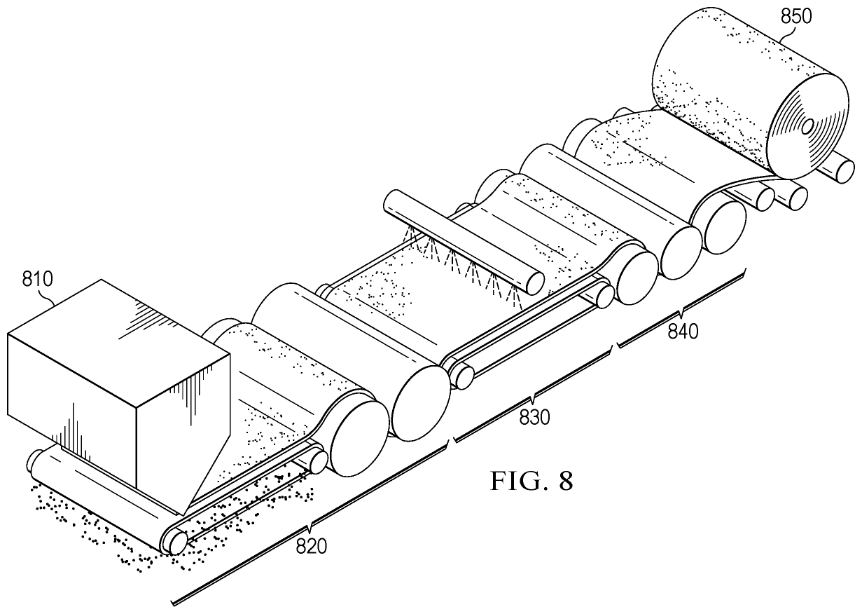

The absorbent cores may comprise one, two, or more of high loft central nonwoven sheet. An absorbent core comprising two high loft central nonwoven sheets is discussed further below with reference to FIG. 7.

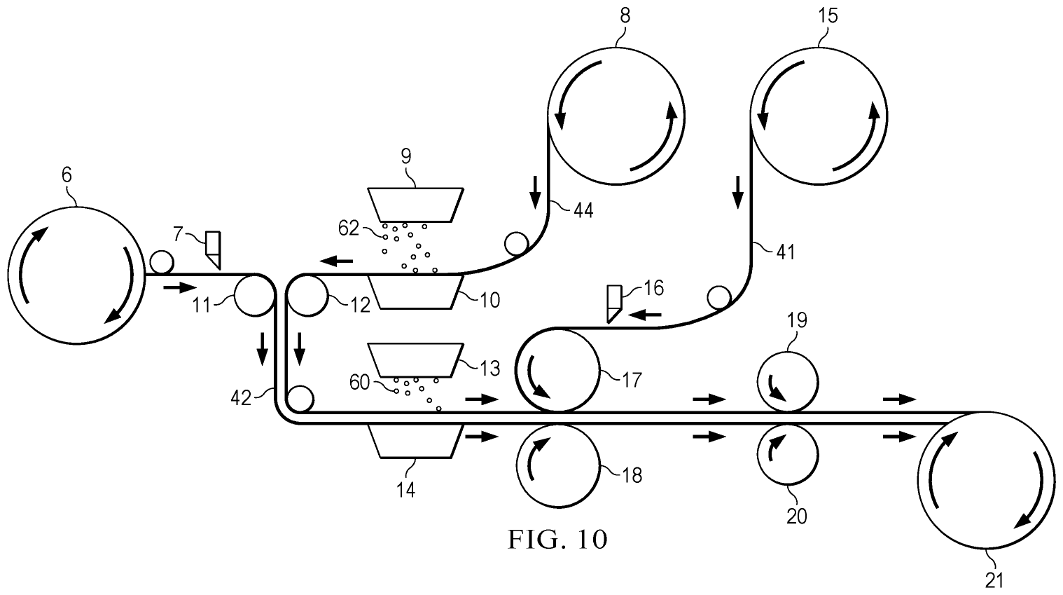

The central nonwoven sheet (or sheets) serves as substrate for the SAP particles 60, 62, 64 which are at least partially distributed within its pores. The SAP particles may be substantially uniformly blended across the thickness of the central nonwoven sheet. However, the SAP particles may be distributed heterogeneously in the vertical direction. The SAP particles are typically deposited on one side of the nonwoven and drawn into the nonwoven for example by gravity or a negative pressure on the opposite side of the nonwoven. In this way, some particles remain close to the surface of the central nonwoven sheet and other, typically smaller, particles may penetrate deeper within the pores of the nonwoven sheet. The SAP particles which are not trapped within the pores of the central nonwoven sheet but remain at the surface may be further immobilized by an optional layer of adhesive 71 or 72. The adhesive may be applied on the top and bottom cover layers first before being combined while still tacky with the central nonwoven sheet. Typically the SAP particles are applied sequentially on the central nonwoven sheet from each side of the central nonwoven sheet as a first SAP layer comprising SAP 60 and a second SAP layer comprising SAP 62, as further illustrated in FIG. 10. This process for SAP particle deposition may result in a SAP z-distribution pattern inside the central nonwoven sheet comprising two or more peaks of density separated by at least one buffer zone, when seen in the z direction.

### Superabsorbent Polymer Particles

The term “superabsorbent polymer” (herein abbreviated as “SAP” in the singular and plural form) typically refers to absorbent materials that can absorb at least 10 times their weight of an aqueous 0.9% saline solution as measured using the Centrifuge Retention Capacity (CRC) test (EDANA method NWSP 241.0.R2 (19)), referred herein as capacity.

SAP are water-insoluble but water-swellable cross-linked polymers capable of absorbing large quantities of fluids. SAP are in particulate form so as to be flowable in the dry state. Typical particulate SAP are polyacrylate polymers, however it is not excluded that other polymer materials may also be used. For example, starch-based particulate absorbent polymer material may also be used, as well polyacrylamide copolymer, ethylene maleic anhydride copolymer, cross-linked carboxymethylcellulose, polyvinyl alcohol copolymers, cross-linked polyethylene oxide, and starch grafted copolymer of polyacrylonitrile.

SAP may be polyacrylates and polyacrylic acid polymers that are internally and/or surface cross-linked. The superabsorbent polymer for the absorbent core of the present invention may be selected from polyacrylates and polyacrylic acid polymers that are internally and surface cross-linked. The superabsorbent polymers can be internally cross-linked, i.e. the polymerization is carried out in the presence of compounds having two or more polymerizable groups which can be free-radically copolymerized into the polymer network. Exemplary superabsorbent polymer particles of the prior art are for example described in WO2006/083584, WO2007/047598, WO2007/046052, WO2009/155265, WO2009/155264. Preferably, the SAP particles comprise crosslinked polymers of polyacrylic acids or their salts or polyacrylates or derivatives thereof.

The absorbent core of the invention, when it comprises an optional central nonwoven sheet, comprises a first SAP layer comprising a first SAP (“SAP1”), and a second SAP layer comprising a second SAP (“SAP2”). Referring to FIGS. 3-6, the first SAP layer comprising SAP1, SAP 60 herein, is close to the top cover layer 41, and the second SAP layer comprising SAP2, SAP 62 herein, is close to the bottom cover layer 42.

SAP1 constituting the first SAP layer and SAP2 constituting the second SAP layer may be the same type of SAP. Or, SAP1 constituting the first SAP layer and SAP2 constituting the second SAP layer may be different types of SAP.

When the absorbent core of the present invention comprises an optional central nonwoven sheet, the SAP particles are typically deposited sequentially on each side of the central nonwoven sheet, with the central nonwoven sheet side being laminated and flipped in between the depositions. The SAP particles can at least partially penetrate within the pores of the central nonwoven sheet during the deposition step, so that they are at least partially distributed within the central nonwoven sheet. The particles are thus immobilized within the pores of the central nonwoven sheet on one hand, and by a top cover layer or a bottom cover layer laminated on each of the top and bottom surface of the central nonwoven sheet.

The total amount of SAP present in the absorbent core may vary according to expected user of the article. The total amount of all SAP in the core may be for example comprised from about 2 g to 50 g, in particular from 5 g to 40 g, or for 10 g to 20 g for typical enfant diapers.

The absorbent core may typically comprise at least 60% by weight of superabsorbent polymer particles (all SAP added), preferably at least 70%, by total weight of the core.

The absorbent cores 28 discussed previously comprises a single optional central nonwoven sheet, however it is also possible that the absorbent cores comprise two (or more) central nonwoven sheets between the top and bottom cover layers. This is illustrated for example in FIG. 7, wherein an absorbent core 28b comprising a first central nonwoven sheet 441 and the second central nonwoven sheet 442 are shown sandwiched between the top cover layer 41 and the bottom cover layer 42.

The absorbent core 28b thus may comprise a first central nonwoven sheet 441 and a second central nonwoven sheet 442, each of which comprising for example superabsorbent polymer particle layers 60, 62, 64 at least partially distributed within the pores of the central nonwoven sheets. The two (or more) central nonwoven sheets may be comprised of the same material or different nonwovens. For example, the permeability in the upper central nonwoven sheet may be enhanced by using a low basis weight nonwoven and softness may be enhanced in the bottom cover layer with a denser nonwoven material. Of course, other configurations are also possible. The two or more two central nonwoven sheets can be of equal dimensions in the X, Y plane of the core, but they may also have different length and/or width. Two central nonwoven sheets of unequal length could be beneficial to provide different amount of SAP along the absorbent core.

At least one of the two central nonwoven sheets comprises two different types of SAP as discussed above, the first SAP 60 having a higher capacity than the second SAP. For example, with the first SAP may be placed in the SAP layer closest towards the top cover layer of the absorbent core and the second SAP 62 and/or 64 may be placed in one of the underlying layer, referring to FIG. 7.

Components of the absorbent articles described herein may at least partially be comprised of bio-based content as described in U.S. Pat. Appl. No. 2007/0219521A1. For example, the superabsorbent polymer component may be bio-based via their derivation from bio-based acrylic acid. Bio-based acrylic acid and methods of production are further described in U.S. Pat. Appl. Pub. No. 2007/0219521 and U.S. Pat. Nos. 8,703,450; 9,630,901 and 9,822,197. Other components, for example nonwoven and film components, may comprise bio-based polyolefin materials. Bio-based polyolefins are further discussed in U.S. Pat. Appl. Pub. Nos. 2011/0139657, 2011/0139658, 2011/0152812, and 2016/0206774, and U.S. Pat. No. 9,169,366. Example bio-based polyolefins for use in the present disclosure comprise polymers available under the designations SHA7260™, SHE150™, or SGM9450F™ (all available from Braskem S.A.).

An absorbent article component may comprise a bio-based content value from about 10% to about 100%, from about 25% to about 100%, from about 40% to about 100%, from about 50% to about 100%, from about 75% to about 100%, or from about 90% to about 100%, for example, using ASTM D6866-10, method B.

### Channel

The absorbent core 28 may comprise at least one longitudinally extending zones substantially free of SAP particles. These one or more zones substantially free of SAP particles are referred to herein as “channels”.

By “substantially free of SAP”, it is meant that the basis weight of the SAP material in each of these zones is at least less than 25%, in particular less than 20%, in particular less than 10%, of the average basis weight of the SAP in the central nonwoven sheet as a whole. The channel may in particular be zones of the central nonwoven sheet where there are no SAP particles. In this regard, minimal amount such as involuntary contaminations with SAP particles that may occur during the making process are not considered as absorbent material.

By “longitudinally extending”, it is meant that the channels extend more in the longitudinal direction (y) than in the transversal direction (x). The channels may be oriented parallel to the longitudinal direction. However, it is not excluded that the channels may be curved, in particular concave towards the longitudinal axis, or straight and tilted at an angle relative to the longitudinal direction.

### Process for Absorbent Core Making

An exemplary continuous process for making the absorbent cores is illustrated in FIG. 10. The process and apparatus discussed above is generally similar to the one disclosed in CN101797201A or in FIG. 3 of WO2020/025401 (BASF, Ge et al.). The various arrows in FIG. 10 represent the rotational directions of the various roll-releasing cylinders and roll-winding cylinders and the running directions of the manufacturing materials during the production flow process. Other processes and modifications are of course possible.

As illustrated in FIG. 10, the apparatus for making the absorbent cores may include a bottom cover layer web unwinder 6, a bottom cover layer glue spraying head 7, a central nonwoven sheet web unwinder 8, a first SAP particles dispenser 9 and optional vacuum suction box 10, a first pair of rollers 11 and 12, a second SAP particles dispenser 13 and optional vacuum suction box 14, a top cover layer web unwinder 15, a top cover layer spraying head 16, second pair of rollers 17 and 18, trimming off knives 19, 20, and a product roll winding roller 21.

The first and second SAP particles dispenser 9, 13 may be both provided with a frequency changing and speed adjusting device (not drawn in FIG. 8), adjusted to maintain a vibration frequency that matches the product roll winding roller 21 linear velocity and to ascertain that the deposited SAP is mostly uniformly distributed on the high loft web 43.

During production, a roll of bottom cover layer material 42, for example a paper or nonwoven roll, is installed on the bottom cover layer web unwinder 6. A high loft nonwoven fabric roll 44 is installed on the central nonwoven sheet web unwinder 8. The initial density and thickness of the high loft sheet may be conveniently measured on the raw material Thickness and Density Test further described below.

The SAP particles are charged in the first and second SAP particles sieve plates 9 and 13. A roll of top cover layer material 41, which can be a paper or nonwoven roll, is installed on the top cover layer web unwinder 15. During the continuous process of making the absorbent cores, the bottom cover layer 42 passes through the spraying head 7 and is applied on one side with a glue 72, before being attached to the central nonwoven sheet 44 between the first press rollers 11 and 12. The high loft nonwoven central nonwoven sheet 44 passes through the first SAP dispenser 9 and vacuum suction box 10, wherein the SAP particles 62 are deposited into the central nonwoven sheet and at least partially distributed into the fibers the central nonwoven sheet from a first side.

After the bottom cover layer 42 and the central nonwoven sheet 44 have been pressed together between the rollers 11 and 12, these combined layers may optionally pass between a second SAP particles sieve plate 13 and vacuum suction box 14 that cooperate to deposit SAP particles 60 onto the second surface of the central nonwoven sheet and blend the SAP particles in the fibers of the central nonwoven sheet from this second surface. The top cover layer 41 which has been applied with an adhesive 72 by a glue spraying head 16 is then joined to the central nonwoven sheet to cover the second surface of the central nonwoven sheet between two press rollers 17 and 18. Of course in the preceding the top cover layer and bottom cover layer may be used interchangeably.

The press rollers 17 and 18 may have a substantially flat surface, or they may have elevated areas where extra pressure and heat should be applied onto the core. These elevated areas may coincide with channel zones in the core, and thus provide a mechanical bonding, ultrasonic bonding and/or heat bonding within the channel zones. The press rollers 11-12, 17-18 may be heated. It is also possible that the rollers have elevated areas along the longitudinal side edges and/or the back and front edges (360° perimeter) the core. A better bonding can be achieved in these zones when they are free of SAP as in the channel zones 26. Trimming knives 19 and 20 can be provided to trim the longitudinal side edges of the continuous band of absorbent core before the stream of the absorbent core material is finally rolled into a roll of absorbent core material by the product roll winding roller 21.

The roll of absorbent core material thus formed may be stored or transported to an article production site where it is further converted into an absorbent product. It is also possible that instead of forming a roll, the stream of absorbent core material may be directly fed into a converting line, in which case the absorbent cores will be individualized by cutting along their front and back edges.

A wrapping layer 3 (not represented in FIG. 10) may also be fed before the core material is rolled to wrap the top, central and bottom cover layer as shown discussed in relation to FIG. 6 to prevent losses of SAP though the side edges of the absorbent core. Alternative such wrapping layer may also be attached to the core when further converting the core material web.

Absorbent cores comprising dual central nonwoven sheets may be made by a method adapted from one of the methods disclosed above, see for example WO2016/106021A1 where two separate central nonwoven sheets releasing cylinders are described to provide for the first and second central nonwoven sheets 441, 442. As an alternative, a central nonwoven web having a double width may be used: such a large width roll can be cut after release in machine direction in two halves providing for two streams of high loft nonwoven material that are then separately deposited with SAP particles. The two streams of central nonwoven sheet material 441, 442 may be then combined separately with the top cover layer and bottom cover layer respectively, each having then SAP particles deposited onto them through a suitable SAP deposition device.

### Absorbent Article 20

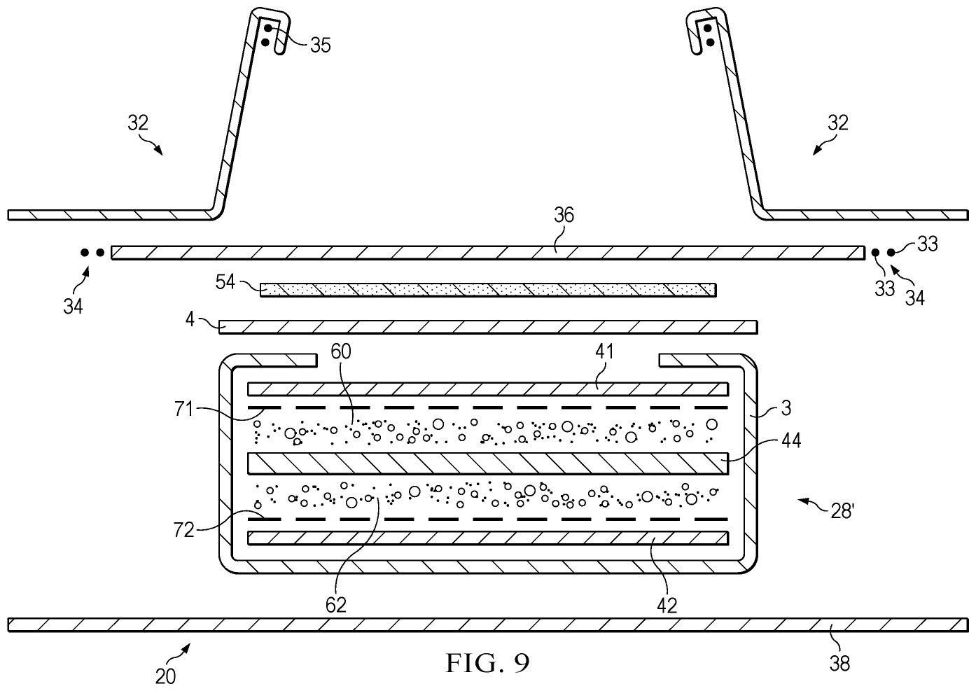

The absorbent cores may be incorporated into any kind of personal hygiene articles, in particular pant diapers and taped diapers, as well as inserts in hybrid systems comprising a washable outer cover and a disposable insert. A schematic cross-sectional view showing some of the main components of a diaper absorbent article 20 is illustrated in FIG. 9. In FIG. 9, the absorbent core of FIG. 6 (with a wrapping layer 3) is shown, but this is of course not limiting and for illustration only. Absorbent articles typically comprise a wearer-facing fluid permeable topsheet 36 and a garment-facing liquid impermeable backsheet 38 attached to each other along their perimeter. The absorbent core is placed between these layers and may be attached directly and indirectly to these layers, typically by gluing or heat/pressure bonding.

The topsheet 36 is preferably compliant, soft-feeling, and non-irritating to the wearer's skin. Further, at least a portion of the topsheet is liquid permeable, permitting liquids to readily penetrate through its thickness. A suitable topsheet may be manufactured from a wide range of materials, such as porous foams, reticulated foams, apertured plastic films, or woven or nonwoven materials of natural fibers (e.g., wood or cotton fibers or viscose), synthetic fibers or filaments (e.g., polyester or polypropylene or bicomponent PE/PP fibers or mixtures thereof), or a combination of natural and synthetic fibers. If the topsheet includes fibers, the fibers may be spunbond, carded, wet-laid, meltblown, hydroentangled, or otherwise processed as is known in the art, in particular spunbond PP nonwoven. Typical diaper topsheets have a basis weight of from about 10 gsm to about 28 gsm, in particular between from about 12 gsm to about 18 gsm but other basis weights are possible.

The backsheet 38 is typically impermeable to liquids (e.g. urine). The backsheet may for example be or comprise a thin plastic film such as a thermoplastic film having a thickness of less than about 0.10 mm. Suitable backsheet materials may include breathable materials which permit vapors to escape from the absorbent article while still preventing exudates from passing through the backsheet. A covering low basis weight nonwoven may be attached to the external surface of the film to provide for a softer touch.

The absorbent articles may also comprise a liquid management layer 54 (also called fluid acquisition or fluid distribution layer) directly under the topsheet 36. The function of such a layer is to rapidly acquire the fluid from the topsheet away from the wearer-facing side and/or to distribute over a larger area so it is more efficiently absorbed by the absorbent core. It is also possible that such a liquid management layer may be placed between the backsheet and the absorbent core. A further layer 4 may be present between the liquid management 54 and the absorbent core 28. The further layer 4 may be another such acquisition or distribution layer, or may be a tissue paper or low basis weight NW layer that provides an additional wrapping of the absorbent core 28′ to avoid SAP particles from escaping outside the core.

Absorbent articles such as diapers or training pants may typically further comprise components that improve the fit of the article around the legs of the wearer, in particular barrier leg cuffs 32 and gasketing cuffs 34. The barrier leg cuffs may be formed by a piece of material, typically a nonwoven, which is partially bonded to the rest of the article and can be partially raised away and thus stand up from the plane defined by the topsheet. The barrier leg cuffs are typically delimited by a proximal edge joined to the rest of the article, typically the topsheet and/or the backsheet, and a free terminal edge intended to contact and form a seal with the wearer's skin. The standing up portion of the cuffs typically comprise an elastic element, for example one or a plurality of elastic strands 35. The barrier leg cuffs provide improved containment of liquids and other body exudates approximately at the junction of the torso and legs of the wearer.

In addition to the barrier leg cuffs, the article may comprise gasketing cuffs 34, which are formed in the same plane as the chassis of the absorbent article, in particular which may be at least partially enclosed between the topsheet or the barrier leg cuffs and the backsheet, and may be placed laterally outwardly relative to the upstanding barrier leg cuffs. The gasketing cuffs can provide a better seal around the thighs of the wearer. Usually each gasketing leg cuff will comprise one or more elastic string or elastic element 33 comprised in the chassis of the diaper for example between the topsheet and backsheet in the area of the leg openings.

The absorbent articles may also include other typical components found in diapers, training pants, replaceable inserts or adult incontinence products (and not further represented). A releasable fastening system for taped diapers may be provided to provide lateral tensions about the circumference of the absorbent article to hold the absorbent article on the wearer. This fastening system is not necessary for training pants since the waist region of these articles is already bonded. The fastening system usually comprises a fastener such as tape tabs, hook and loop fastening components, interlocking fasteners such as tabs & slots, buckles, buttons, snaps, and/or hermaphroditic fastening components, although any other known fastening means are generally acceptable. A landing zone is normally provided on the front waist region of the article for the fastener to be releasably attached.

The absorbent article may comprise front cars and back cars as is known in the art. The cars can be integral part of the chassis, for example formed from the topsheet and/or backsheet as side panel. Alternatively, they may be separate elements attached by gluing and/or heat embossing. The back cars are advantageously stretchable to facilitate the attachment of the tabs on the landing zone and maintain the taped diapers in place around the wearer's waist. The front ears may also be elastic or extensible to provide a more comfortable and contouring fit by initially conformably fitting the absorbent article to the wearer and sustaining this fit throughout the time of wear well past when absorbent article has been loaded with exudates since the elasticized cars allow the sides of the absorbent article to expand and contract.

Typically, adjacent layers will be joined together using conventional bonding method such as adhesive coating via slot coating or spraying on the whole or part of the surface of the layer, or thermo-bonding, or pressure bonding or combinations thereof. The bonding between components is for clarity and readability not represented in the majority of Figures, in particular FIG. 9, except for adhesive layers 71, 72. Adjacent layers of the article should be considered to be attached to another unless specifically mentioned otherwise. For example the backsheet and the bottom cover layer of the absorbent core may be typically glued together. The adhesives used may be any standard hotmelt glue as known in the art.

### Measurement

### 1. Basis Weight Test

The basis weights of materials are measured according to WSP 604.0 (08). All measurements are performed in a laboratory maintained at 23° C.±2 C.° and 50%±2% relative humidity and test specimens are conditioned in this environment for at least 2 hours prior to testing. To obtain the nonwoven sample, cut a rectangle-shaped nonwoven specimen from the article with an area of 100 cm² (for example, 100 mm×100 mm), and measure its basis weight following the measurement principle used by the standard method above. At least 5 replicates for the test specimen are tested and the average value of the at least five replicates is reported to the nearest 1 gsm (g/m2) as the basis weight of the test specimen.

### 2. Thickness and Density Test

This test is used to measure the thickness (caliper) of nonwoven in a standardized manner. The density can then be calculated from the thickness and the basis weight of the layer. Unless otherwise mentioned, the thickness and density are indicated for the high loft material in the absence of SAP particles. The measurement should preferably be made on the high loft material before it was converted into an absorbent core and thus free of SAP. If the starting material is not available, the high loft central nonwoven sheet can be obtained by carefully extracting it from an absorbent core, and removing the majority of SAP particles for example by careful shaking or suction. A freeze spray may be used to separate the central nonwoven sheet from the other layers. The samples should be kept at least 24 hours at 21° C.±2° C. and 50%±10% RH to equilibrate, in particular if they have been previously compressed.

Equipment: Mitutoyo manual caliper gauge with a resolution of 0.01 mm, or equivalent instrument.

Contact Foot: Flat circular foot with a diameter of 16.0 mm (±0.2 mm). A circular weight may be applied to the foot (e.g., a weight with a slot to facilitate application around the instrument shaft) to achieve the target weight. The total weight of foot and added weight (including shaft) is selected to provide the desire pressure, for example 4.14 kPa of pressure (0.6 psi) to the sample. The thickness can be determined at different pressures, using accordingly different weights applied to the foot. The thickness and density measurements indicate the applied pressure, for example measured at 4.14 kPa (0.6 psi) or 1.2 kPa.

The caliper gauge is mounted with the lower surface of the contact foot in a horizontal plane so that the lower surface of the contact foot contacts the center of the flat horizontal upper surface of a base plate approximately 20×25 cm. The gauge is set to read zero with the contact foot resting on the base plate.
Ruler: Calibrated metal ruler graduated in mm.
Stopwatch: Accuracy 1 second.

Sample preparation: The central nonwoven sheet is conditioned at least 24 hours as indicated above.

Measurement procedure: The layer is laid flat with the bottom side, i.e. the side intended to be placed towards the backsheet in the finished article facing down. The point of measurement, i.e. the middle of the sample, is carefully drawn on the top side of the layer, taking care not to compress or deform the layer. In the unlikely case that the high loft nonwoven layer is not homogeneous in the transversal direction or longitudinal direction, the values are measured in the center of a sample corresponding to the center of an absorbent core that would be made from the sample.

The contact foot of the caliper gauge is raised and the central nonwoven sheet is placed flat on the base plate of the caliper gauge with the top side of the core up so that when lowered, the center of the foot is on the marked measuring point.

The foot is gently lowered onto the sample and released (ensure calibration to “0” prior to the start of the measurement). The caliper value is read to the nearest 0.01 mm, 10 seconds after the foot is released.

The procedure is repeated for each measuring point. Ten samples are measured in this manner for a given material and the average thickness is calculated and reported with an accuracy of one tenth mm. The basis weight of each sample is calculated by dividing the weight of each sample by their area.

The density, in g/cm³, is calculated by dividing the basis weight (in g/cm²) of the material by the thickness (in cm).

### 3. Tensile Strength Test

MD tensile strength of a specimen is measured according to NWSP 110.4-09 with conditions below.
Test Speed: 100 mm/min
Sample Width: 50 mm
Sample length: sufficiently longer than gauge length
Gauge Length: 100 mm

### 4. Wicking Length Test

A wicking length in a machine direction of a nonwoven is measured according to ISO 9073-6:2000 “Textiles—Test methods for nonwovens—Part 6: Absorption 6. Liquid wicking rate”. (EN29073 part 6)

When a nonwoven to be tested is available in a raw material form, a specimen with a size of 30 mm×250 mm is cut from the raw material. When a nonwoven is a component of an absorbent core or a finished product, the nonwoven is removed from the core or the finished product using a razor blade to excise the nonwoven from other components of the finished product to provide a nonwoven specimen with a size of 30 mm×250 mm is cut from the raw material. A cryogenic spray (such as Cyto-Freeze, Control Company, Houston TX) may be used to remove the nonwoven specimen from other components of the finished product, if necessary.

4 replica for each sample are tested and an average value (arithmetic mean) of the 4 replica is reported as a MD wicking length.

### 5. Wicking Area Test

This method measures the XY distribution area by free dropping 0.1 ml test liquid on a test material such as nonwoven. Test liquid, 0.9% NaCl with dye color, was prepared by dissolving 45 g NaCl in 2500 ml deionized water and dyeing the solution obtained with a colorant.

When a nonwoven to be tested is available in a raw material form, a specimen with a size of 100 mm×100 mm is cut from the raw material. When a nonwoven is a component of an absorbent core or a finished product, the nonwoven is removed from the core or the finished product using a razor blade to excise the nonwoven from other components of the finished product to provide a nonwoven specimen with a size of 100 mm×100 mm is cut from the raw material. A cryogenic spray (such as Cyto-Freeze, Control Company, Houston TX) may be used to remove the nonwoven specimen from other components of the finished product, if necessary.

### Procedure

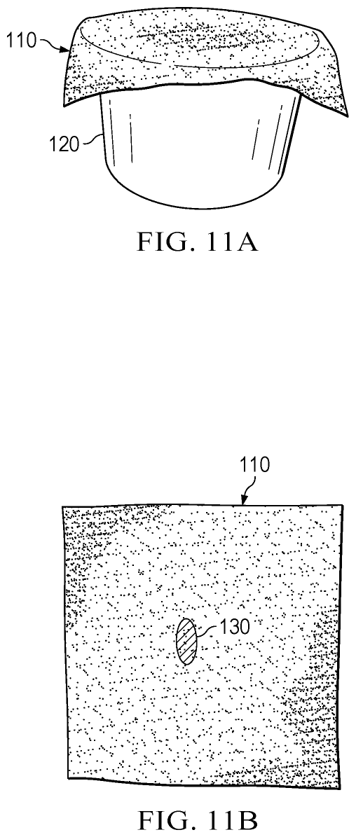

1. Cut a specimen with a size of 100 mm×100 mm is cut from a raw material or an absorbent core.
2. Put a cylindrical bracket with a circle cavity (diameter 6-8 cm) on a bench top.
3. Referring to in FIG. 11A, place the specimen on the bracket flatly in such a way that the center of the specimen is placed on the center of circle cavity of the bracket.
4. Drop 10 ul test liquid in the center of the specimen. At this moment start a stopwatch.
5. Remove the specimen from the bracket after 1 min, and place it on a bench top.
6. Draw an outline of a wicking area, a colored area, on the specimen, referring to FIG. 11B.
7. Measure the wicking area by image J or VHX digital microscope, or equivalent software program at a proper magnification to capture the entire wicking area.
8. Repeat the steps 1-7 for 4 additional replica. Averages of the wicking areas from 5 specimens is reported as a wicking area of the specimen.

### 6. Modified Fluid Acquisition Test

The Modified Fluid Acquisition (“MFA”) Test is designed to measure the speed at which 0.9% saline solution is absorbed into an absorbent article that is compressed at 2.07 kPa. A known volume is introduced four times, each successive dose starting five (5) minutes after the previous dose has absorbed. Times needed to absorb each dose are recorded. The test fluid is 0.9% w/v saline solution and is prepared by weighing 9.0 g±0.05 g of NaCl into a weigh boat, transferring it into a 1 L volumetric flask, and diluting to volume with de-ionized water.

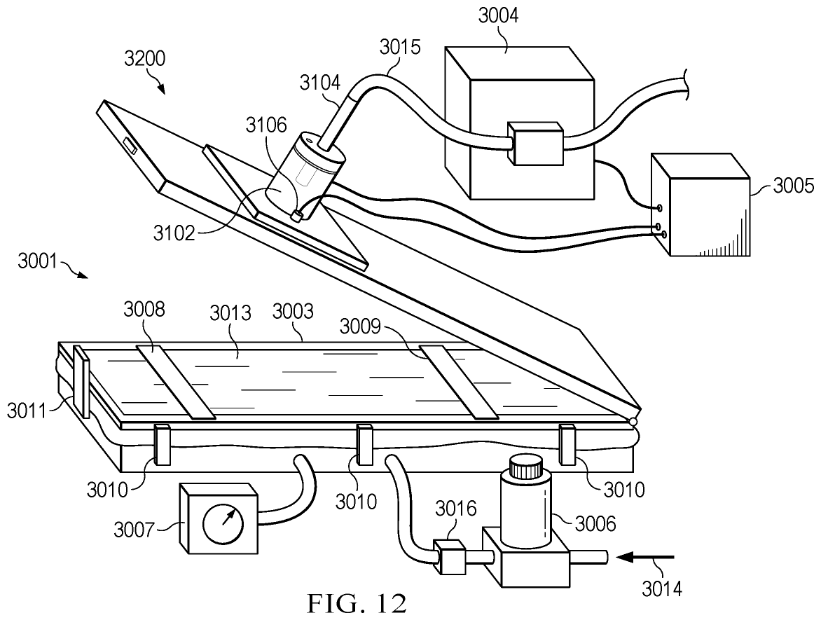

The MFA apparatus is depicted in FIG. 12 through FIG. 14B. The MFA apparatus comprises a bladder assembly 3001 and a top plate assembly 3200 that includes a deposition assembly 3100. A controller 3005 is used to 1) monitor the impedance across electrodes 3106, recording the time interval 0.9% saline solution is in a cylinder 3102, 2) interface with a liquid pump 3004 to start/stop dispensing, and 3) time intervals between dosing. The controller 3005 is capable of recording time events to ±0.01 sec. A house air supply 3014 is connected to a pressure regulator 3006 capable of delivering air at a suitable flow/pressure to maintain 2.07 kPa in the bladder assembly 3001. A liquid pump 3004 (Ismatec MCP-Z gear pump, available from Cole Palmer, Vernon Hills, IL or equivalent) capable of delivering a flow of 10-80 mL at a rate of 3-15 mL/s is attached to a steel tube 3104 of the deposition assembly 3100 via tygon tubing 3015.

The bladder assembly 3001 is constructed of 12.7 mm Plexiglas with an overall dimension of 80 cm long by 30 cm wide by 10 cm tall. A manometer 3007 to measure the pressure inside the assembly and a pressure gauge 3006 to regulate the introduction of air into the assembly are installed through two holes through the light side. A bladder 3013 is assembled by draping a 50 mm by 100 mm piece of silicone film, (thickness 0.02″, Shore A durometer value of 20, available as Part #86435K85 from McMaster-Carr, Cleveland, OH) over the top of the box with enough slack that the film touches the bottom of the box at its center point. An aluminum frame 3003 with a flange is fitted over the top of the film and secured in place using mechanical clamps 3010.

When in place, the assembly should be leak free at a pressure of 3.45 kPa. A front 3008 and back 3009 sample support of 5 cm by 30 cm by 1 mm are used to anchor the sample. The absorbent article is attached to the top surface of the sample supports by either adhesive tape or mechanical “hook” fasteners. These supports can be adjusted along the length of the aluminum frame 3003 via a simple pin and hole system to accommodate different size absorbent articles and to correctly align their loading point.

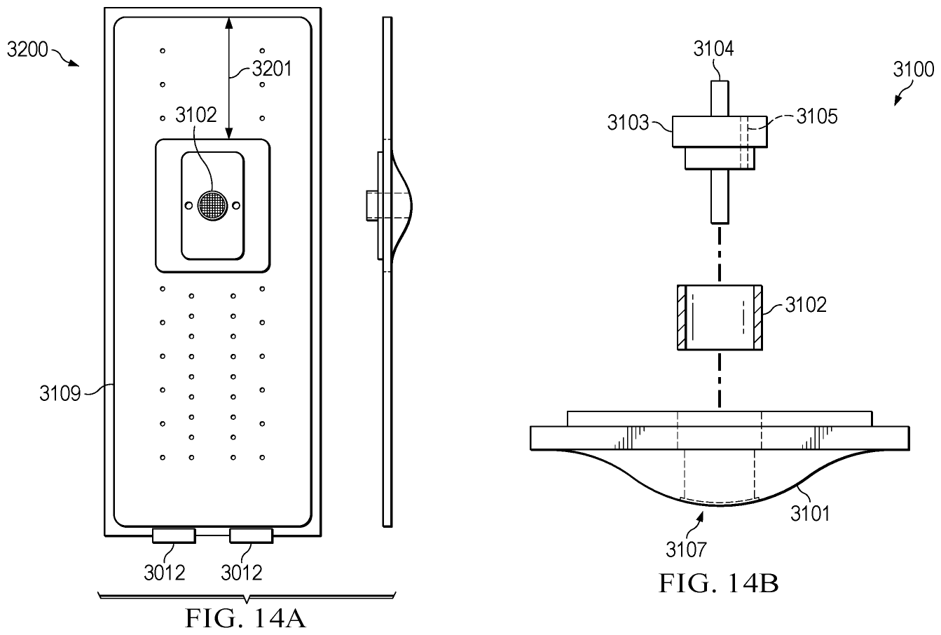

The top plate assembly 3200 is constructed of an 80 cm by 30 cm piece of 12.7 mm Plexiglas reinforced with an aluminum frame 3109 to enhance rigidity. The plate has a cutout 170 mm wide by 201 mm long centered laterally on the plate, 170 mm from the front of the plate 3201 for mounting of the deposition assembly. In addition, the top plate has thirty-six (36) 3.2 mm diameter holes drilled through it distributed as shown in FIG. 14A. The holes prevent air from being trapped under the top plate as the bladder is inflated. The top plate assembly 3200 is connected to the bladder assembly 3001 via two hinges 3012. During use, the top assembly is closed onto the bladder assembly and locked into place using a mechanical clamp 3011.

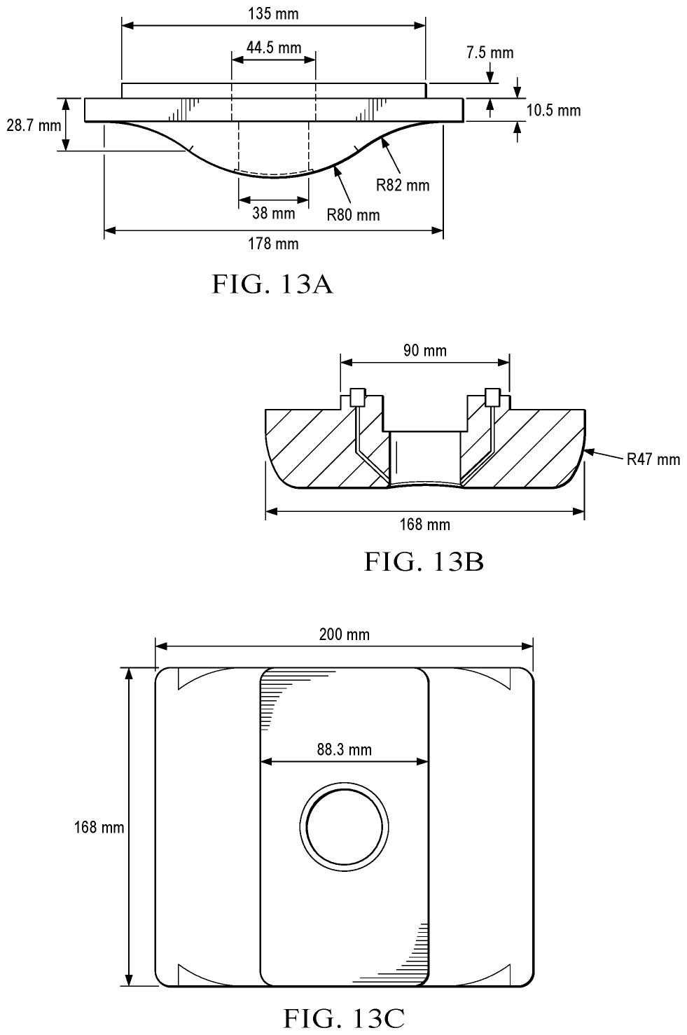

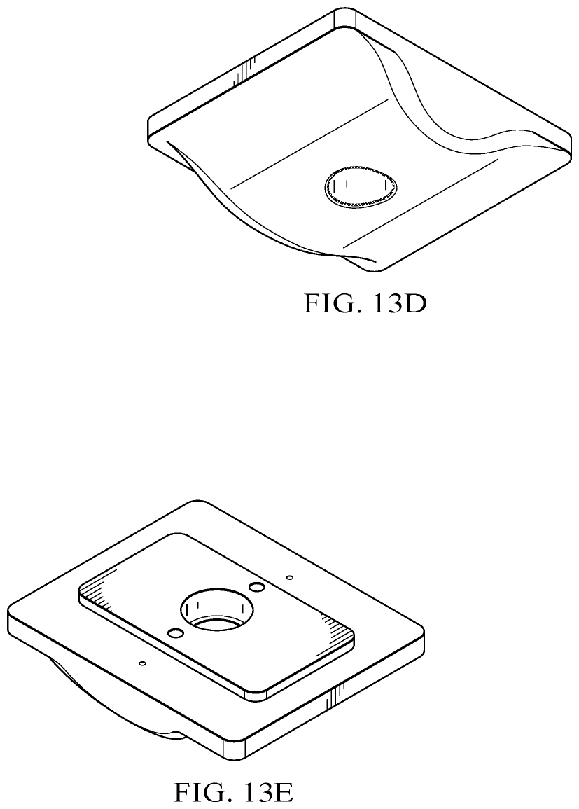

The deposition assembly 3100 is fitted into the top plate 3200 and includes 1) a liquid introduction cylinder 3102, 2) a curved surface 3101 at the loading point of the absorbent article and 3) electrodes 3106 that are used to detect fluid in the cylinder 3102. The detailed dimensions of the curved component are provided in FIG. 13A to FIG. 13E. FIG. 13A is a side view of the curved component. FIG. 13B is an end view of the curved component. FIG. 13C is a bottom view of the curved component. FIG. 13D is a bottom perspective view of the curved component. FIG. 13E is a top perspective view of the curved component. This curved component can be milled or 3D printed. The top portion of the introduction cylinder is a 50.8 mm O.D. Plexiglas cylinder 3102 with a 38.1 mm LD. This is fitted into the curved component to give the introduction cylinder a total height of 100 mm. Imbedded electrodes run from connectors on the upper surface of the curved component and terminate flush with an inside wall of the introduction cylinder, 2 mm from the bottom of the cylinder. The two electrodes are positioned 180 degrees apart. A nylon screen 3107 is cut and affixed flush with the bottom of the cylinder such that the sample cannot swell into the cylinder. A 5 mm semi-circle is cut in the screen in the immediate area of the two electrodes. The deposition assembly is inserted into the top plate as shown in FIG. 14A such that the curved surface is flush with the bottom of the top-plate assembly 3200. The introduction cylinder 3102 is topped with a loose-fitting nylon cap 3103. The cap has a 6.35 mm O.D. steel tube 3104 inserted through its center. When the cap is in place, the bottom of the tube ends 20 mm above the screen 3107. The cap also has an air hole 3105 to ensure negative pressure does not impede the absorption speed.

The absorbent article is first prepared by excising any inner or outer leg cuffs, waist caps, elastic ears or side panels, taking care not to disturb the topsheet that resides above the article's core region. Place the absorbent article flat onto a lab bench and identify the intersection of the longitudinal centerline with the size dependent loading point as defined in Table 1.

TABLE 1
Conditions for Modified Fluid Acquisition Testing:

| | | Loading Point from | | Single | |
|---|---|---|---|---|---|
| | Approximate | front of Core* | | Dose | Flow |
| Diaper | Baby Weight | Boy | Girl | Volume | Rate |
| Size | Pounds | mm | mm | mL | mL/s |
| 1 | 8 to 13 | 64 | 64 | 24 | 8 |
| 2 | 13 to 17 | 76 | 89 | 24 | 8 |
| 3 | 17 to 28 | 89 | 114 | 50 | 10 |
| 4-6 | 28+ | 102 | 127 | 75 | 15 |

*the boy loading point is used for unisex diapers.

Attach the front end of the absorbent article to the top surface of the front sample plate 3008 by either adhesive tape or mechanical “hook” fasteners with a topsheet facing upward. The placement is such that just the chassis and not the absorptive core overlays the plate. The sample plate 3008 is attached to the aluminum frame 3003 such that the size-dependent Loading Point (as defined in Table 1) of the absorbent article will be centered longitudinally and laterally within the cylinder 3102 when the top plate assembly has been closed. The back end of the absorbent article is secured to the back sample plate 3009 by either adhesive tape or mechanical “hook” fasteners, once again ensuring that only the chassis and not the absorptive core overlays the plate. The back sample plate 3009 is then attached to the aluminum frame 3003 such that the article is taunt but not stretched. The top plate assembly is closed and fastened, and the bladder is inflated to 2.07 kPa±0.07 kPa. The pressure is maintained at this level during the complete loading sequence of the test.

The pump 3004 is primed and then calibrated to deliver the size-dependent volume and flow rate selected from Table 1. Volume and flow rate must be within ±2% of target. The cap 3103 is placed into the cylinder 3102. The controller 3005 is started, which in turn delivers the first dose of 0.9% saline solution. After the volume has been absorbed, the controller waits for 5.0 minutes before addition of the next dose. This cycle is repeated for a total of four doses. If the fluid leaks out of or around the article (i.e., is not absorbed into the article) then the test is aborted. Also if any acquisition time exceeds 1200 seconds, the test is aborted. The acquisition time is defined as the difference between the start time (i.e., when the 0.9% saline is first introduced into the cylinder and that conducting fluid completes the circuit between the electrodes) and the stop time (i.e., when the fluid has completely drained from the cylinder and the circuit between the electrodes is broken). Acquisition times are recorded by the controller for each dose to the nearest 0.01 second. After the last dose is acquired, pressure is applied for an additional 10 minutes. Open the pressure relief valve 3016 to deflate the bladder and then remove the sample from the acquisition system.

In like fashion, run a total of eight (8) replicates for each absorbent article to be evaluated. Calculate and report the Acquisition Times (sec) for each dose as the arithmetic mean of the replicates to the nearest 0.01 sec.

### 7. Rewet Test

The Rewet Test is performed immediately after the MFA Test. The Rewet Test comprises measuring the mass of fluid expressed from an absorbent article under pressure after loading by the MFA protocol. Filter paper is used as the rewet substrate. A suitable filter paper is Whatman™ 125 mm diameter (Cytiva Cat No. 1001-125) or equivalent. Upon receipt, the filter paper stored at about 23° C.±2 C.° and about 50%±2% relative humidity for 2 hours prior to testing. Equipment for the test consists of a Plexiglas disk 70.0 mm in diameter and a stainless steel confining weight that rests upon it. The mass of the disk and confining weight combined is 812.5 g which corresponds to a pressure of 2.07 kPa. Filter papers are put together as the stacks of three (3) assembled for use during rewet testing. Measure and record the mass of the dry filter paper stack and record to the nearest 0.001 g.

Within 30 seconds after the conclusion of the MFA test, remove the absorbent article from the acquisition apparatus and place it flat on a bench top with a topsheet facing upward. Then, place a pre-weighed stack of filter paper centered at the loading point (as determined previously in the MFA test), place the Plexiglass disk onto the stack, and gently place the confining weight onto the disk. Wait for 15.0 seconds±0.5 seconds and remove the weight and disc. Immediately measure the mass of the wet filter paper and record to the nearest 0.001 g. Calculate the filter paper value as the difference between the wet and dry weight of the stack and record to the nearest 0.001 g.

In like fashion, run a total of eight (8) replicates for each absorbent article to be evaluated. Calculate and report the Collagen Rewet (mg) for each dose as the arithmetic mean of the replicates to the nearest 0.001 g.

### EXAMPLES

### Example 1: Nonwoven

MD wicking length and XY wicking area were tested on various nonwovens according to test methods disclosed herein and are displayed in Table 2 below.

TABLE 2

| | Nonwoven 1 | Nonwoven 2 | Nonwoven 3 | Tissue 1 | Tissue 2 | Nonwoven 4 |
|---|---|---|---|---|---|---|
| Production | Wetlaid | Wetlaid | Wetlaid | Wetlaid | Wetlaid | Spunlace |
| Process | | | | | | |
| Composition | 60% pulp, | 50% pulp, | 50% pulp, | 100% | 100% | 100% |
| | 40% viscose | 50% viscose | 50% viscose | pulp | pulp | viscose |
| Basis weight | 40 | 45 | 57 | 25 | 50 | 45 |
| (gsm) | | | | | | |
| MD wicking | 139 | 114 | 130 | | | 113 |
| length (mm) | | | | | | |
| MD wicking | 3.48 | 2.53 | 2.28 | | | 2.51 |
| length /basis | | | | | | |
| weight(mm/gsm) | | | | | | |
| XY wicking | 331 | 289 | 240 | | | 262 |
| area(mm²) | | | | | | |
| MD tensile | 19.07 | 49.2 | | 5.1 | 6.9 | |
| strength (N) | | | | | | |
| MD tensile | 0.48 | 1.09 | | 0.20 | 0.15 | |
| strength/basis | | | | | | |
| weight (N/gsm) | | | | | | |

| | Nonwoven 5 | Nonwoven 6 | Nonwoven 7 | Nonwoven 8 | Nonwoven 9 |
|---|---|---|---|---|---|
| Production | Spunlace | Airlaid | Spunlace | Spunlace | Spunbond |
| process | | | | | |
| Composition | 100% | 70% pulp, | 30% viscose, | 50% viscose, | Polypropylene |
| | viscose | 30% PE/PET* | 70% PET | 50% PET | |
| Basis weight | 40 | 46 | 35 | 35 | 23 |
| (gsm) | | | | | |
| MD wicking | 129 | 51 | 12.25 | 48.5 | 0 |
| length (mm) | | | | | |
| MD wicking | 3.23 | 1.11 | 0.35 | 1.39 | 0 |
| length/basis | | | | | |
| weight(mm/gsm) | | | | | |
| XY wicking | 294 | 59.1 | 53.3 | 66.5 | 78.2 |
| area(mm²) | | | | | |

Nonwoven 1, and Nonwovens 2 and 3 are produced by different manufacturers.
*Polyethylene/polyethylene terephthalate sheath/core bicomponent fibers

Nonwovens 1-3 comprise pulp at least 30% by weight of the nonwoven, and have high MD wicking length values and a high MD tensile strength. Nonwovens 1-3 have MD tensile strength much higher than 12N, and MD tensile strength/basis weight much higher than 1.5N/gsm.

### Example 2: Absorbent Core

Absorbent cores using various top cover layers and bottom cover layers, a common central nonwoven sheet, and commercially SAP were produced according to Table 3 below. In all cases, the central nonwoven sheet was a 40 gsm air through bonded carded nonwoven having a density of 0.018 g/cm³ and a caliper of 2.1 mm measured at 2.07 kPa.

TABLE 3

| | Core 1 | Core 2 | Core 3 | Core 4 |
|---|---|---|---|---|
| Top cover layer | Nonwoven 1 | Nonwoven 7 | Nonwoven 8 | Nonwoven 9 |
| SAP1 (SAP 60) (gsm) | 176 | 178.5 | 178.5 | 178.5 |
| Central nonwoven sheet | 40 gsm high loft NW | | | |
| SAP2 (SAP 62) (gsm) | 176 | 178.5 | 178.5 | 178.5 |
| Bottom layer | Nonwoven 9 | Nonwoven 8 | Nonwoven 7 | Nonwoven 8 |

### Example 3: Absorbent Article

Baby diapers 1-4 as exemplary absorbent articles having absorbent cores in Example 2 above were fabricated using a common topsheet, distribution layer, and backsheet. Baby diapers 5-8 as exemplary absorbent articles having absorbent cores in Example 2 above were fabricated using a common topsheet, distribution layer, and backsheet.

Acquisition time and rewet of each of Diapers 1-8 were measured according to Acquisition Speed Test and Rewet Test disclosed herein, and results are indicated in Table 4 below.

TABLE 4

| | | Diaper 1 | Diaper 2 | Diaper 3 | Diaper 4 | Diaper 5 | Diaper 6 | Diaper 7 | Diaper 8 |
|---|---|---|---|---|---|---|---|---|---|
| Topsheet | | 41 gsm carded air through | | | | 35 gsm, carded air through | | | |
| | | nonwoven from 2D PE/PET | | | | nonwoven from 2.6D eccentric PE/PET | | | |
| Core | | Core 1 | Core 2 | Core 3 | Core 4 | Core 1 | Core 2 | Core 3 | Core 4 |
| Acquisition | 75 ml | 86 | 91 | 81 | 88 | 91 | 89 | 80 | 90 |
| time (sec) | 75 ml | 197 | 236 | 193 | 247 | 202 | 217 | 183 | 264 |
| | 75 ml | 403 | 487 | 354 | 528 | 389 | 417 | 312 | 574 |
| Rewet (g) | | 0.053 | 0.128 | 0.104 | 0.187 | 0.059 | 0.136 | 0.078 | 0.116 |

Diapers 1 and 5 having absorbent cores according to the present invention, compared to Diapers 2-4 and 6-8, respectively, exhibit a significantly fast acquisition speed, and significantly low rewet.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular examples of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An absorbent core for use in an absorbent article, the absorbent core extending in a transversal direction and a longitudinal direction and having a thickness in a vertical direction perpendicular to the transversal direction and longitudinal direction, the absorbent core comprising:
a liquid-permeable top cover layer;
a bottom cover layer; and
an absorbent layer disposed between the top cover layer and the bottom cover layer, the absorbent layer comprising superabsorbent polymer particles,
wherein the top cover layer comprises pulp, and has an MD tensile strength at least about 12N, wherein the top cover layer has a first MD wicking length at 3 min, and the bottom cover layer has a second MD wicking length at 3 min as measured according to Wicking Length Test, wherein the first MD wicking length is at least about 70 mm, and at least about 3 times greater than the second MD wicking length.

2. The absorbent core of claim 1, wherein the first MD wicking length is at least about 100 mm.

3. The absorbent core of claim 1, wherein the absorbent layer further comprises a central nonwoven sheet having a top surface oriented towards the top cover layer and a bottom surface oriented towards the bottom cover layer, wherein at least part of the superabsorbent polymer particles is deposited on at least one of the top surface and the bottom surface of the central nonwoven sheet.

4. The absorbent core of claim 3, wherein the central nonwoven sheet comprises a high loft carded nonwoven.

5. The absorbent core of claim 1, wherein the absorbent layer is substantially free of free cellulose fibers.

6. The absorbent core of claim 1, wherein the top cover layer comprises absorbent fibers in a concentration of at least about 80% by weight of the top cover layer.

7. The absorbent core of claim 1, wherein the top cover layer comprises pulp and viscose fibers.

8. The absorbent core of claim 1, wherein the top cover layer comprises pulp in an amount of at least about 30% by weight of the top cover layer.

9. The absorbent core of claim 1, wherein the top cover layer consists essentially of absorbent fibers.

10. The absorbent core of claim 1, wherein the top cover layer further comprises synthetic fibers.

11. The absorbent core of claim 1, wherein the top cover layer comprises wetlaid nonwoven material.

12. The absorbent core of claim 1, wherein the top cover layer has a first wicking area, wherein the first wicking area is at least about 100 mm².

13. The absorbent core of claim 1, wherein the bottom cover layer comprises synthetic fibers.

14. The absorbent core of claim 1, wherein the bottom cover layer comprises spunbond nonwoven material.

15. The absorbent core of claim 1, wherein the absorbent core further comprises at least one longitudinally-extending channel substantially free of superabsorbent polymer particles.

16. An absorbent article comprising a topsheet, a backsheet, and an absorbent core disposed between the topsheet and the backsheet, wherein the absorbent core is one according to claim 1.

17. An absorbent core for use in an absorbent article, the absorbent core extending in a transversal direction and a longitudinal direction and having a thickness in a vertical direction perpendicular to the transversal direction and longitudinal direction, the absorbent core comprising:
a liquid-permeable top cover layer;
a bottom cover layer; and
an absorbent layer comprising superabsorbent polymer particles,
wherein the top cover layer comprises first absorbent fibers and second absorbent fibers, the second absorbent fibers being different from the first absorbent fibers, and
wherein the total amount of the first absorbent fibers and the second absorbent fibers is at least about 70% by weight of the top cover layer.

18. The absorbent core of claim 17, wherein the absorbent layer further comprises a central nonwoven sheet having a top surface oriented towards the top cover layer and a bottom surface oriented towards the bottom cover layer, wherein at least part of the superabsorbent polymer particles is deposited on at least one of the top surface and the bottom surface of the central nonwoven sheet.

19. The absorbent core of claim 17, wherein the top cover layer comprises pulp in an amount of at least about 30% by weight of the top cover layer.

20. The absorbent core of claim 19, wherein the top cover layer comprises an MD tensile strength at least about 12N.
